(19) United States
(12) Patent Application Publication
(10) Pub. No.: US 2017/0023509 A1
(43) Pub. Date: Jan. 26, 2017

(54) CROWDSOURCED WEARABLE SENSOR SYSTEM

(71) Applicant: CHEMISENSE, INC., Berkeley, CA (US)

(72) Inventors: BRIAN KIM, BERKELEY, CA (US); DEV MEHTA, BERKELEY, CA (US); WILLIAM HUBBARD, BERKELEY, CA (US); AMRIT KASHYAP, BERKELEY, CA (US); MICHAEL KEATON, BERKELEY, CA (US); WOO YONG CHOI, BERKELEY, CA (US); GEENA KIM, BERKELEY, CA (US)

(21) Appl. No.: 15/277,766

(22) Filed: Sep. 27, 2016

Related U.S. Application Data
(63) Continuation of application No. PCT/US2015/025787, filed on Apr. 14, 2015.
(60) Provisional application No. 61/980,004, filed on Apr. 15, 2014.

(51) Int. Cl. G01N 27/12

(52) U.S. Cl. CPC G01N 27/126; G01N 33/0034

(57) ABSTRACT

The present invention provides devices, systems and methods for effective chemical detection. The technology is applicable across many industries, including personal respiratory health, mining safety, food processing, and defense. In certain aspects, the devices, systems and methods of the present invention allow for environmental gas detection to be used for respiratory disease sufferers.

### CROSS-REFERENCES TO RELATED APPLICATIONS

The present application is a continuation of PCT/U.S. 2015/025787, filed Apr. 14, 2015, which claims priority to U.S. Patent Application No. 61/980,004, filed Apr. 15, 2014, the teachings of which are hereby incorporated by reference in their entireties for all purposes.

### BACKGROUND OF THE INVENTION

The accelerating trend of connected devices has led to a marked increase in the demand for low cost, portable and accurate sensors across a wide range of industries. In parallel with the pursuit for more data in almost every sector of the economy to improve efficiency, decision-making and outcomes, the broad availability of cloud computing enables businesses to apply and action insights from their growing data sets.

Despite advances in sensor technology to address new demands, chemical detection is one area that has not seen advances that change the logistics of cost, size, power and sensitivity required for an emerging connected economy. Today, product-focused technology companies can measure everything from acceleration to temperature using a sensor easily integrateable into a cell phone, but currently there is no reliable method to determine the presence of certain chemicals on or around the environment of users and their devices.

Indoor and outdoor air pollution is directly responsible for the deaths of 3.3 million people each year. It is also directly or indirectly responsible for a wide range of chronic health and lifestyle issues, ranging from asthma to COPD. Clearly, significant health benefits can be realized by monitoring and controlling exposure to air pollution, but current solutions in both the industrial and consumer spaces are either lacking or non-existent. For example, over 26 million Americans suffer from asthma, and over 56 billion dollars each year is spent combatting the worst effects of the disease. Yet, by providing an air quality monitor to avoid disease triggers, the quality of life of the individual patients could see immense benefits.

In view of the foregoing, there is a need in the art for a wearable sensor system that gives real time data regarding air quality. The present invention satisfies these and other needs.

BRIEF SUMMARY OF THE INVENTION

The present invention provides a crowdsourced wearable sensor system for air quality monitoring applications. As such, in one embodiment, the present invention provides a wearable sensor system for air quality monitoring, the wearable sensor system comprising:
a module comprising an array of chemiresistors;
a microcontroller with a wireless transmitter; and
a signal generator.

In another embodiment, the present invention provides a method for detecting an analyte with a wearable sensor system, the method comprising:
contacting an analyte with a wearable sensor system, the wearable sensor system comprising a module having an array of chemiresistors; a microcontroller with a wireless transmitter; and a signal generator; and
detecting an electrical change in the array of chemiresistors in the presence of the analyte.

These and other aspects, objects and advantages will become more apparent with the detailed description and drawings which follow.

### BRIEF DESCRIPTION OF THE DRAWINGS

FIGS. 1A-B illustrate (A) one embodiment of a wearable sensor; and (B) a schematic of a sensor of the present invention.

FIGS. 2A-C illustrate (A) one embodiment of a wearable sensor of the present invention; (B) an exploded view of a wearable sensor; and (C) a wearable sensor of the present invention.

FIGS. 3A-C illustrate (A) one embodiment of a wearable sensor; (B) an embodiment of sensor network; and (C) an embodiment of sensor communication.

FIGS. 4A-B illustrate (A) one embodiment of a mesh network; and (B) an embodiment of sensor network of the present invention.

FIGS. 5A-D illustrates (A) one embodiment of a sensor device; (B) one embodiment of a sensor device; (C) one embodiment of a sensor device; and (D)one embodiment of a sensor device.

FIG. 6 illustrates schematics of a prototype. The square is a 1 cm×1 cm chemiresistor film sprayed onto four electrodes.

FIG. 7 illustrates analyte exposure (ethanol) to a chemiresistor made of polyvinyl stearate, PVA, and P4VP. The solid lines are moving averages to show a trend.

FIGS. 8A-B illustrate analyte exposure (acetic acid) to a chemiresistor array of the present invention.

FIGS. 9A-B illustrate analyte exposure (toluene) to a chemiresistor array of the present invention.

FIGS. 10A-B illustrates analyte exposure (tetrahydrofuran) to a chemiresistor array of the present invention.

FIG. 11A-B illustrates analyte exposure to a chemiresistor array of the present invention.

FIG. 12 illustrates a cross-section of a substrate of a chemiresistor array of the present inventions with an applied chemiresistor film.

FIGS. 13A-F illustrate analyte exposure to a chemiresistor array of the present invention. FIGS. 13A-C illustrate exposure of toluene a chemiresistor made of PEVA at concentrations of (A) 100 ppm, (B) 50 ppm, and (C) 50 ppm. FIG. 13D illustrates exposure of acetic acid to a chemiresistor made of PEO. FIG. 13E illustrates exposure of toluene to a chemiresistor made of PEO. FIG. 13F illustrates exposure of acetic acid to a chemiresistor made of PEVA. FIG. 13G illustrates exposure of heptane to a chemiresistor made of PEVA. FIG. 13H illustrates exposure of benzene to a chemiresistor made of P4VP.

FIG. 14 illustrates a chemiresistor array of the present invention containing rows of polymers and columns of analytes.

### DETAILED DESCRIPTION OF THE INVENTION

### I. Embodiments

The present invention provides devices, systems and methods for low-cost and effective chemical detection. The technology is applicable across many industries, including personal respiratory health, mining safety, food shipping and air quality monitoring. In certain aspects, the devices, systems and methods of the present invention allow for environmental gas detection to be used for asthmatics and other respiratory disease sufferers.

In one aspect, the present invention provides a sensor system that stays with a user whether indoors or outdoors, and senses air contaminates (analytes) or gases in real time. When a user enters an area with poor air quality, or if a change in air quality is detected, the sensor warns the user by sending an alert to the user's smartphone, and triggering a signal by the signal generator such as a vibration or audible warning at the device. In certain aspects, the system comprises crowdsourced data from nearby users, thus making the measurements and resulting data more robust.

In one embodiment, the present invention provides a wearable sensor system for air quality monitoring, the wearable sensor system comprising:
a module comprising an array of chemiresistors;
a microcontroller with a wireless transmitter; and
a signal generator.

In certain aspects, the present invention provides a crowdsourced wearable sensor system for personal air quality monitoring applications. The present invention takes advantage of a plurality of individuals wearing the sensors (crowdsourced), which are each multiple data points for air quality monitoring. “Crowdsourcing” is the process of obtaining the needed data from a large group of people wearing the sensors of the present invention. By utilizing many data points in a specific geographic area, it is possible to cover the area with more sensors than if only a single individual and a single sensor were used. By covering a specific area, the density of sensors per unit area is high. Thus, the data is very reliable.

In certain aspects, the density of wearable sensor units is variable. For example, in a metropolitan area, such as New York City or Los Angeles, the density is about 1 wearable sensor per every about 100 square meters, (meter²/wearers).

In a rural area the density can be less. In such areas, the density is about 1 wearable sensor per every about 2000 square meters, (meter²/wearers). The reasons why a much lower density for rural areas is feasible is at least two-fold. The first is the relative dearth of point sources of pollution. For example, if a user is in a city, there are far more cars, factories, etc. all of which can create localized sources of pollution. However, with far less of those sources in rural areas, fewer sensors per a given area are needed. The second is that the air flow within rural areas is far less restricted. Indeed, a user in an area with a large number of high rises or skyscrapers, air flow within the area is restricted to channels between buildings, and it is harder to get a larger sample of air. However, in much flatter rural areas, air flow is far less restricted, allowing for better mixing, necessitating far fewer sensors per unit area. As a rural area changes, more sensors per unit area are added.

In certain aspects, the data derived from the crowdsourced sensors is hyperlocal. For example, in one aspect, 50 or more individuals wearing the sensors of the present invention are within a city or county boarder or a particular zip code. This number of deployed sensors allows far more granular data than current available technology, especially when compared to the current status quo of air monitoring stations established in some cities. A user with a subject device has truly hyperlocal data, and this data is generalized for some distance around the user depending on environmental factors such as those mentioned above. Further, a single sensor is sufficient to get data on air quality within a given region, but by having many tens of devices, the resulting data from each individual sensor is crosschecked against any or all of the others in the region, allowing for greater accuracy than otherwise possible.

In certain other aspects, the present invention provides a wearable sensor to individuals such as employees of a confined area such as a refinery, an oil field or pilot plant. The wearable sensors provide real time data for both indoor and outdoor air quality levels.

In certain other instances, by using the devices, systems and methods of the present invention, it is possible to obtain real time alerts. Thus, it is no longer necessary to wait for public broadcasts on radio, TV or the internet. Using the crowdsourced systems of the present invention, such alerts come from the present methods and wearable sensor systems.

### A. Chemiresistor Array

Chemiresistors of the present invention work on the principal of absorption and desorption. When an analyte is detected by the chemiresistor, it is adsorbed onto a carbon film, which can be impregnated by a variety of compounds such as polymers. Once the analyte concentration decreases, the adsorbed analyte will then desorb as the concentration gradient of the analyte moves away from the film.

The present invention provides an array of sensors having at least two sensors, wherein each of the least two sensors is compositionally the same or different. The sensors are preferably chemiresistors, each chemiresistor having electrical leads. There exists an electrical path across the sensor, or between the electrical leads.

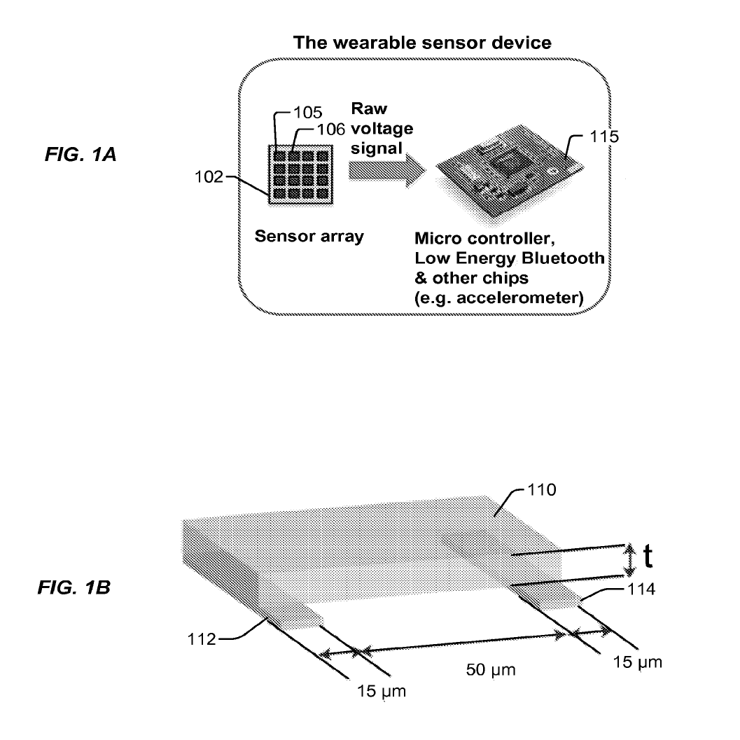

FIG. 1A illustrates one embodiment of a sensing device disposed in a wearable device of the present invention. In this illustrative example, the sensor array 102 is a 4×4 array with 16 different sensors. Each of the 16 sensors has a different polymer or different amount (e.g., concentration) of polymer make up or composition. The device also comprises a multi-chip module (MCM) 115 including a microcontroller. Examples of an MCM include, but are not limited to, a printed circuit board with prepackaged integrated circuits, a chip stack with multiple integrated circuits, and a custom chip package on a high density interconnection substrate. The microcontroller may be used to process an electrical change (e.g., voltage changes, resistance changes, impedance changes, combinations of these and the like) in each of the sensors. The MCM may also include several other chips, including but not limited to, memory, accelerometer, power supply and device controller, voltage regulator, Bluetooth, Wi-Fi, microprocessor, battery charger, and a gyroscope.

In one instance, the first sensor in the array 105 is different than the second sensor 106 in the array. For example, the first sensor comprises a first polymer and the second sensor comprises a different polymer. In another instance, the first sensor and the second sensor comprise the same polymer, however each sensor comprises different concentrations of the polymer. In one instance, each sensor comprises a polymer and carbon black.

A variety of polymers are suitable for use in the manufacture of the sensors of the present invention. The polymer can be a conducting polymer, a nonconducting polymer or a mixture thereof. The polymer can be mixtures of polymers. Suitable polymers are disclosed in

U.S. Pat. No. 5,571,401, incorporated herein by reference in its entirety for all purposes.

Suitable polymers include, but are not limited to, poly(dienes), poly(alkenes), poly(acrylics), carbon polymers poly(methacrylics), poly(vinyl ethers), poly(vinyl thioethers), poly(vinyl alcohols), poly(vinyl ketones), poly(vinyl halides), poly(vinyl nitrites), poly(vinyl esters), poly(styrenes), poly(arylenes), poly(oxides), poly(carbonates), polylesters), acyclic heteroatom poly(anhydrides), poly(urethanes), polymers poly(sulfonates), poly(siloxanes), poly(sulfides), poly(thioesters), poly(sulfones), poly(sulfonamides), poly(amides), poly(ureas), poly(phosphazenes), poly(silanes), poly(silazanes), poly(furan tetracarboxylic acid diimides), heterocyclic poly(benzoxazoles), poly(oxadiazoles), polymers poly(benzothiazinophenothiazines), poly(benzothiazoles), poly(pyrazinoquinoxalines), poly(pyromenitimides), poly(quinoxalines), poly(benzimidazoles), poly(oxindoles), poly(oxoisoindolines), poly(dioxoisoindolines), poly(triazines), poly(pyridazines), poly(piperazines), poly(pyridines), poly(piperidines), poly(triazoles), poly(pyrazoles), poly(pyrrolidines), poly(carboranes), poly(oxabicyclononanes), poly(dibenzofurans), poly(phthalides), poly(acetals), poly(anhydrides), carbohydrates, poly(halohydrins), and thermoplastic polymers, and mixtures thereof.

In one aspect, each chemiresistor comprises a polymer and carbon black.

In one aspect, the polymer is a cellulosic polymer. Advantageously, cellulosic substrates are porous and effectively enlarge the surface area exposed by the chemiresistors to air, and by extension, enlarge the effective detection area of the sensor compared to other substrates. Moreover, the cellulosic sensors have excellent signal to noise.

In one aspect, each chemiresistor comprises a polymer and carbon black. In one aspect, an electrical pattern (e.g., voltage, resistance, or impedance signal pattern) is produced from the array of chemiresistors and is collected by the microcontroller. In certain aspects, the signal pattern is processed by an algorithm (principal component analysis (PCA)) to detect and or identify a gas i.e., analyte. In one instance, each polymer responds differently to each chemical or contaminate gas i.e., analyte, such that the combination of the signals from the array can be unique to the specific analyte.

In certain aspects, the algorithm (e.g., PCA) is resident on the microcontroller of the multi-chip module 115.

Other sensor technologies suitable for use in the present systems and devices include, but are not limited to, semiconductor sensors (e.g., metal oxide, polysilicon, etc), solid or gel electrolyte gas sensors, piezoelectric gas sensors (e.g., SAW, FBAR, Quartz oscillator), conductive polymers, optical fiber or waveguide sensors (such as being based on a change of an optical property when gas is absorbed by the material), ChemFET, chemiresitors and a combination thereof.

In certain instances, the sensor array produces a given pattern of resistances like an aggregate of resistances indicative of the analyte. Thus, for a given analyte or vapor, a sensor array will produce a unique pattern of resistances for that analyte. The pattern can be stored on board, in a mobile device, or on a server (e.g., a remote server). In this manner, a library of patterns is generated and stored. The pattern formed by the wearable sensor i.e., the unknown pattern can be compared to stored patterns in a library of patterns. The unknown response pattern can be identified through a comparison algorithm such as PCA.

In one instance, the electrical pattern (e.g., voltage, resistance, or impedance signal pattern) from the sensor array are collected frequently, such as about 1 to 10,000 seconds or about 1 to 300 minutes and the data is processed by an algorithm (for example, principal component analysis) on a chip to identify the analyte and the concentration of the analyte. A display or indicator can show the indication/threat level (e.g. ambient, low/med/high, harmful) and the class of chemical triggering the alert (e.g. NO₂ or SO₂). If the air quality dips below a certain point, the device can be made to further alert the user via text, vibrations or sound or signal. In certain aspects, the device connects to another mobile system, such as a smart phone.

In certain instances, the sensor array is in a cartridge or module. In certain instances, the sensor array within the cartridge or module has been optimized for a particular analyte or vapor. For example, an asthmatic is susceptible to certain VOCs such as NO and other gases that are irritating. By optimizing the sensors within the cartridge to these analytes, it is possible for the asthmatic to be prepared to detect an analyte, vapor, or gas in which they are susceptible.

In operation, the sensor array imbibes the analyte which in turn changes the electrical properties (e.g. resistance, voltage, and the like) and elicits a response pattern. Depending on the analyte, and the polymer of the sensor, each member of the sensor array will imbibe the analyte differently.

In certain instances, the wearable sensor can process the analyte on MCM 115. In other instances, the resistance or voltage pattern is processed on a mobile device or server. The unknown pattern can be compared to the library on the mobile device or remote server. Pattern recognition software can compare the unknown pattern against the library patterns to identify the unknown.

The sensor array comprises at least two sensors and up to 10,000 sensors. In other instances, the array comprises 2, 4, 8, 12, 16, 32, 64, 128 or even more sensors. In some aspects, there is a control sensor, which can be a positive control, a negative control or both.

This can ensure the array is properly tuned with low background.

FIG. 1B shows one embodiment of the present invention. The upper object 110 represents a polymer-carbon composite film with a thickness ‘t’, and the bottom objects 112, and 114 represent an electrode pair made of for example, silver or copper. In one embodiment, the film is about 0.5 mm-long along the electrodes. Four-terminal sensing can also be used.

In certain aspects, the analyte(s) desorb from the sensor based on concentration. Thus, after the analyte is sensed, the concentration gradient moves and the sensors desorb the analyte. However, in certain instances, the sensors need to be purged or cleaned. In order to clean the sensors, and purge them from any residue left by the previous analyte, the sensor(s) can be heated to desorb the previously measured analyte. This heating increases the duty cycle of the sensor array. The sensors can be heated by photo-irradiation or thermal energy to desorb the vapors from the film.

In one aspect, the wearable sensor comprises a miniaturized UV lamp or micro- or nano-thermal heater that is placed in the vicinity of the composite film to radiate and/or conduct energy to desorb the analyte from the film. This returns the array to the baseline voltage and extends the useful lifespan of the chemiresistor.

In certain aspects, the sensor system further comprises one or more of a member selected from the group consisting of an accelerometer, a UV-lamp, a micro-heater, a nano-heater, a GPS module, a temperature sensor, a humidity sensor, an RFID tag, and a battery.

In a preferred aspect, the sensor system comprises an accelerometer. An accelerometer can measure the speed of the user and transmit the data to a smartphone and to the cloud. Based on the speed and movement pattern, user movement information can be inferred. For example, if the user location does not change for long time and the movement is low the user is likely to be indoors therefore the measured air quality data does not contribute the crowdsourced air quality map. Also the speed and movement pattern information helps to infer whether the user is in a car, walking, or exercising.

In one aspect, the sensor module is disposable.

### B. Analytes

A wide variety of analytes are detectable using the sensors of the present invention. In certain instances, the analytes are volatile organic compounds (VOCs). These analytes represent a wide range of potentially dangerous analytes from carcinogens to major air pollutants, in addition to a number of more benign compounds as well.

In certain aspects, the analytes include, volatile organic compounds such as acetone, acetic acid, formaldehyde, benzene, ethanol, and the like. The device detects a variety of non-organic pollutants such as CO, NO₂, NH₃, and the like.

Volatile organic compounds (VOCs) are typically emitted as gases from certain solids or liquids. VOCs include a variety of chemicals, some of which may have short- and long-term adverse health effects. Concentrations of many VOCs are consistently higher indoors (up to ten, fifteen or twenty times higher) than outdoors. VOCs are emitted by a wide variety of products numbering in the thousands. Examples include, but are not limited to, paints, varnishes, lacquers, paint strippers, cleaning supplies, pesticides, insecticides, building materials, furnishings, office equipment such as copiers and printers, correction fluids and carbonless copy paper, graphics and craft materials including glues, adhesives, permanent markers, and photographic solutions.

Other substances emitting VOCs include, cleaning, disinfecting, cosmetic, degreasing, and hobby products. Fuels and gasoline also emit VOCs.

### C. Wristband Device

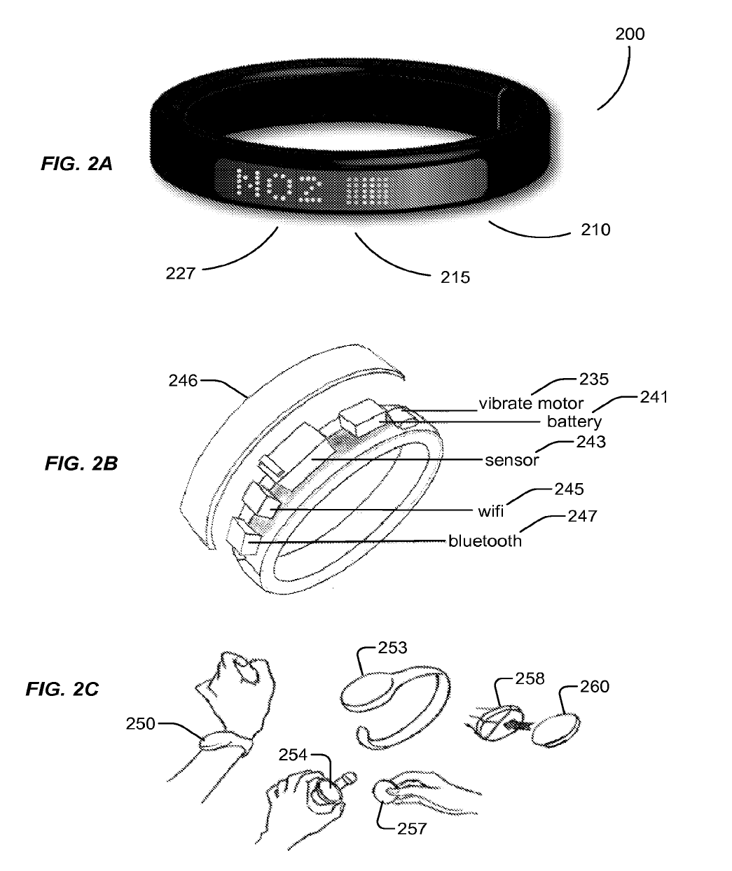

Turning now to FIG. 2A, in certain aspects, the wearable sensor device 200 is disposed within a housing 210 such as a bracelet. The bracelet 210 can be worn by the user like a watch or wrist band. The bracelet 210 has a display 215 that indicates various contaminates in the air and displays the identity of the gas or analyte 227 (e.g. NO₂).

In certain instances, the sensor system for the chemical detection is linearized to meet the requirements of a wristband form factor. In one aspect, this is performed by modifying the circuit layout. Further, the wearable device can also include a flexible display, which cycles the display to show the current gas levels in the surrounding environment.

FIG. 2B shows the display and membrane cover 246 removed and various features such as vibrate motor 235, battery 241, sensor array 243, Wi-Fi 245, and Bluetooth 247. Further, the wearable sensor array has optional USB charging. The battery may be charged wirelessly through induction charging, through an auxiliary connector on the band, or a USB port such as a mini-USB, micro-USB, USB 3.0, USB 3.1 or other USB-type port.

In certain aspects, the wearable device optionally includes one or more of the following an accelerometer, a gyroscope, a temperature sensor, a humidity sensor, low-power Bluetooth module, battery, battery charging module, and a microcontroller. The accelerometer can be used in conjunction with a low power GPS module for activity and location tracking for accurate exposure levels.

In certain instances, the sensing elements are covered by a membrane 246. A wide range of membrane materials exist which provide a physical barrier to a gas and or water-vapor.

The device may also include a shock resistant frame/mesh for making the device strong and robust.

The device may be modular to allow various elements to be replaced over time, including a battery, sensing elements, and if needed, even other components including an accelerometer.

Turning now to FIG. 2C, the wearable sensor is shown on the wrist 250 and off the wrist 253. In addition, a replaceable cartridge 257, 260 is shown. The cartridge 260 or 257 comprising a sensor array can be tailored to specific analytes. For example, analytes like NO₂, and CO are more likely to be found in the atmosphere, but working in an industrial setting, H₂S or other toxic chemicals can be present. Hence, the sensing cartridge for the two applications might be different. It is possible to plug the cartridge 257 or 260 into the wearable device 254, 258 and play the sensors. Thus, the plug and play nature of the sensor cartridge is useful for different sensing applications. A skilled artisan will understand that this is one possible representation of a wearable sensor cartridge, i.e., a circular mountable piece is but one example of the modular/cartridge system.

The cartridge comprises sensing components, along with the hardware for directly connecting it to circuits in the band itself. The electrical responses (e.g., resistance changes) from different sensors are processed and displayed through LED lighting/screen with vibrational and sound alerts. This serves in place of, or in conjunction with the aforementioned flexible display screens.

The air quality levels are displayed on the mobile device or any other monitoring device including, but not limited to, Google glasses, computer monitors, tablets, and the like.

In one embodiment, the housing on which the cartridge is mounted includes all data acquisition, processing and relaying components including Bluetooth, GPS, microcontroller and processor.

### D. Wireless Operation:

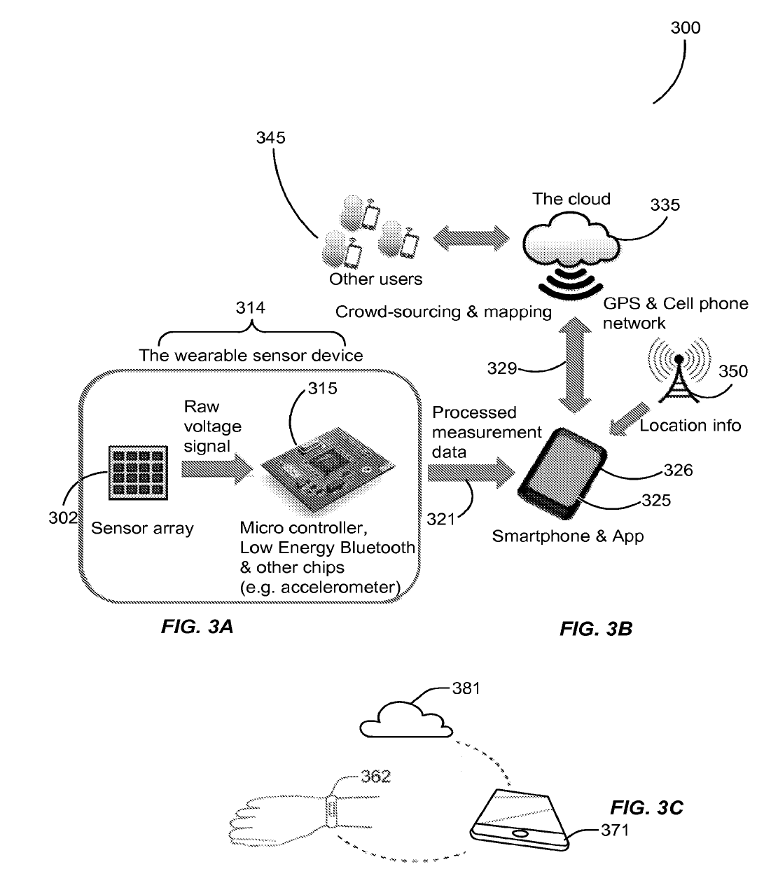

Turning now to FIGS. 3A-C, the normal operation of the wearable air quality sensor device 314 is shown. As described earlier, in one embodiment the sensor array 302 is a 4×4 array with 16 different sensors. The device also comprises a multi-chip module (MCM) 315 including a microcontroller. The microcontroller may be used to process the electrical changes (e.g. voltage changes) in each of the sensors. Examples of an MCM include, but are not limited to, a printed circuit board with prepackaged integrated circuits, a chip stack with multiple integrated circuits, and a custom chip package on a high density interconnection substrate. The MCM may also include several other chips including, but not limited to, memory, accelerometer, power supply and device controller, voltage regulator, Bluetooth, Wi-Fi, microprocessor, battery charger, and a gyroscope.

Turning now to FIG. 3B, the processed measurements 321 are sent to a smartphone 325 having an application 326 that allows the sensor device 314 to communicate to the outside world. In a preferred aspect, the smartphone 325 has an application 326 that allows it to communicate with the wearable sensor device 314 through a Bluetooth transmission protocol. A Bluetooth chip included in MCM 315 of the device can be used to perform this communication. Other forms of wireless communication between the wearable sensor device include, but are not limited to, Wi-Fi, cellular, ANT, UWB, ZigBee, and 6LoWPAN. In addition, the smartphone 325 or mobile device communicates to cloud-based 335 data storage and analysis. In system 300, mobile device 325 executes mobile application 326, which connects with a mobile cloud service (MCS) 335. In certain aspects, communication from the mobile device 325 to MCS 335 can be accomplished using a standalone cloud service (chemisense.com).

Air quality data that is obtained by the sensor array 302 may be communicated through a low energy transmitter (e.g., Bluetooth) of a MCM 315 to a smartphone 325, or via similar wireless communications discussed herein. The data can then be uploaded to the cloud for crowdsourced mapping of air quality. The data synchronization with the cloud can occur every 1-50 seconds or a similar time period. Once the data is synchronized, a heat-map of air quality can be created using cloud computing resources to analyze data stored in the cloud. In the mapping, mathematical models and/or systems are used to estimate effective range and concentration and or distribution of chemical(s) based on measurement at certain location points 350.

In certain aspects, one or more electrical signals (e.g. voltage signals) from the sensor array are transmitted from the wearable sensor 314 to a mobile device, such as a cell phone 325. In certain aspects, the identity of the gas or analyte is transmitted from the wearable sensor 314 to a mobile device 325. For example, the transmission of the voltage signal from the wearable device 314 to a mobile device 325 is via Bluetooth, cellular, Wi-Fi or other wireless technology. In certain aspects, the mobile device is a smartphone, cellular phone, tablet or PC.

In one aspect, the mobile device 325 communicates to a server in the cloud 335. In a preferred aspect, a plurality of mobile devices 345 communicate to a server in the cloud 335. In one aspect, a mathematical system resides on the server to process the incoming data to identify a gas or an analyte

In certain aspects, the server generates a localized map of air quality. Further, an algorithm or mathematical model can be used to estimate a range and concentration of a gas.

In certain aspects, the mobile device 325 receives the identity of the gas from the server in the cloud 335. In one aspect, the mobile device 325 receives and visualizes a localized map from the server 335. In one aspect, a localized map is overlayed on a user's location and may be displayed by mobile device 325.

Turning now to FIG. 3C, in one aspect, the user's movements 362 are transmitted to the server in the cloud 381. In certain aspects, the mobile device 371 transmits the identity of a gas or analyte to the wearable sensor system 362.

In one aspect, a gas is indicative of poor air quality. In certain instances, the signal generator produces a signal such as a light, a sound, heat, a vibration or a combination thereof

If and when the levels of a particular contaminant in the hyperlocal environment contact the sensing elements, the device will sense it, and then relay it to the cloud either via Bluetooth or other wireless communication means provided on MCM 315 to a smartphone.

Depending on the power requirements, the wearable sensor has internal processing capabilities; in that case it can uses Wi-Fi, Bluetooth, or other wireless communication or a combination, to relay the data to a smartphone, monitor, or any other display device, for example a head-mounted display or a smart watch.

After processing the data internally or externally, the wearable device issues a vibrational and/or sound alert to make user aware of any dropping air quality.

The user is also able to learn about the air quality levels on the screen of a mobile phone/tablet/monitor. The system has the capabilities of logging the sensor response from one person and generating real time air quality heat maps.

The system also has the ability to alert users in the immediate vicinity and in areas of poor air quality if and when sensors are triggered.

In certain instances, the system generates exposure level maps (heat maps) of air quality data collected from active devices in real time. The generated map can then be transmitted directly back to the users of the devices themselves.

In another instance, the system tracks lifetime exposure to various gases present in the environment to a user or an entire building/region of the user is a company or government building. The net result is that the solution can be more than just a reactive one; it is proactive as well.

The smartphones or other mobile devices can have various operating systems, such as the Apple iOS, Google Android, or Microsoft Windows Mobile operating systems. The devices run custom-built applications, sometimes referred to as “apps,” for the mobile device. The apps connect through cellular protocols and/or local wireless networks to the Internet.

A smartphone application uses the air quality map data from the cloud, visualizes and displays it. The resulting heat map is downloaded in real time and shown on the application. The user's location information through GPS/AGPS from the smartphone can be overlaid on the map. Using such methods, a user can understand the air quality around him or her and can proactively avoid areas or routes with a bad air quality. Additional features include air quality maps by chemical and historic data of air quality in both picture and graphical forms.

In an alternate aspect, rather than actively sending data from the sensor to the cloud or another device, more passive methodologies are used. For example, in one embodiment, an RFID tag is added to the chip. In another embodiment, the circuit containing the chemiresistor further comprises an RFID tag. An advantage of these embodiments is the elimination of the power requirements of running the sensor. Similar to embodiments that require only enough power to run an electric current through a resistor, embodiments utilizing an RFID tag require a very small amount of power. However, by using the power from an RFID tag reader, no power is required to be supplied by the device itself. Thus, in one aspect, the system comprises smart sensor tags that can be placed in a wider variety of locations and uses where it is impracticable to utilize embodiments requiring power. For example, these smart tags are manufactured via inkjet printing and placed within food containers and/or packages to monitor the various volatile organic compounds given off as the food ages.

### E. Applications

In certain aspects, the present invention provides systems, devices and methods which allow for real time air quality monitoring within a designated local area.

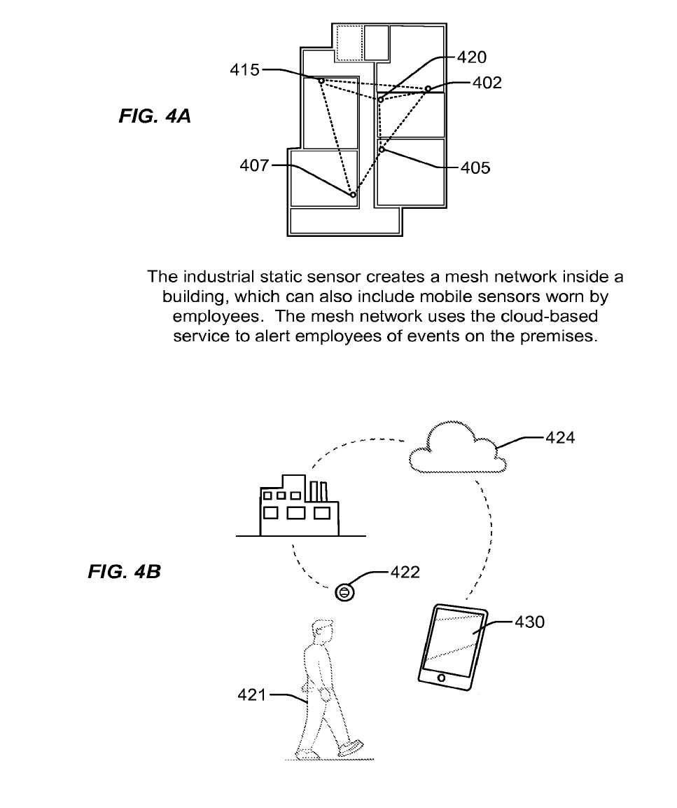

For example, the designated local area can be inside a building such as a school, office building or stadium. As shown in FIG. 4A, an office building can have a plurality of fixed sensors creating a network inside the building. Various sensors 402, 405, 407, 415 and 420 are located throughout the building.

Another example is illustrated in FIG. 4B. The internal network in FIG. 4A can be used by employee 421. Because the internal network is in cloud 424, employee 421 can be alerted via smart phone 430 of a particular chemical threat.

In yet another example, in one aspect, the local area is an oil refinery. Using the wearable sensors of the present invention, it is possible to define the scope of detection with the accessibility of the individual(s) wearing the sensors. As the sensors are networked, it is possible to derive specific air quality at a defined location. The identity of the analyte can be done on board, on a mobile device, or at a remote server.

In another example, analytes detectable by the device of the invention include, but are not limited to, alkanes, alkenes, alkynes, dienes, alicyclic hydrocarbons, arenes, alcohols, ethers, ketones, aldehydes, carbonyls, carbanions, heterocycles, polynuclear aromatics, organic derivatives, biomolecules, microorganisms, bacteria, viruses, sugars, nucleic acids, isoprenes, isoprenoids, and fatty acids and their derivatives. Many biomolecules are amenable to detection using the sensors of the invention.

The wearable device can be used for medical and first responders to quickly and accurately identify the chemical components in the air, on a subject's breath, wounds, and bodily fluids to diagnose a host of illness including infections and metabolic problems. Further, the devices and systems can be used to test for skin conditions, and other ailments. Alternatively, the device can classify and identify microorganisms, a microbiome and bacteria.

The devices and systems can be used in food and fruit quality and processing control. For example, the device can be used to spot test for immediate results or to continually monitor batch-to-batch consistency, ripeness and spoilage in various stages of a product, including production (i.e., growing), preparation, and distribution.

The devices and systems can be used in detection, identification, and/or monitoring of combustible gas, natural gas, H₂S, ambient air, emissions control, air intake, smoke, hazardous leak, hazardous spill, fugitive emission, beverage, food, and agricultural products monitoring and control, such as freshness detection, fruit ripening control, fermentation process, and flavor composition and identification, detection and identification of illegal substance, explosives, transformer fault, refrigerant and fumigant, formaldehyde, diesel/gasoline/aviation fuel, hospital/medical anesthesia, sterilization gas, telesurgery, body fluids analysis, drug discovery, infectious disease detection and breath applications, worker protection, arson investigation, personal identification, perimeter monitoring, HVAC automation in both industrial and civilian settings, tracking of personal respiratory health, tracking of exposures to different pollutants on a personal basis as well as cumulative basis, fragrance formulation, and solvent recovery effectiveness, refueling operations, shipping container inspection, enclosed space surveying, product quality testing, materials quality control, product identification and quality testing.

In one embodiment, the sensor system is used for HVAC automation purposes in industrial applications as well as consumer applications. For example, an air quality sensor array is positioned in the interior of a vehicle and another sensor array is positioned on the exterior of a vehicle such as an automobile. By compiling the data from both sensors, it is possible to compare the air quality on both sides of the vehicle, and thus discern which is healthier for the occupants of the vehicle to be breathing. If one of the occupants begins to smoke on an otherwise clear day, the vehicle automatically opens-up the recirculation in the car's HVAC, allowing the cleaner air that was on the outside of the vehicle to enter. In contrast, if the car is being driven in during a particularly smoggy day, the vehicle closes off the recirculation, ensuring that the comparatively cleaner cabin air quality remains inside the vehicle for as long as possible.

In another embodiment, data and processing centers across the United States need to have the temperature, humidity and air quality levels controlled especially within the rooms containing the data cores themselves. If there is a buildup of any of the three factors mentioned above, severe damage to the centers, as well as any people entering the room could occur. Currently, many centers simply run high power air condoning through these rooms on a 24/7 basis. However, using devices, systems and methods of the present invention, and combining the sensors with a temperature and a humidity sensor as previously described, users see significant cost reductions and benefits by using the sensor to turn on ventilation only when needed rather than running it on a permanent basis. Multiple sensors are deployed for a single data center, and the HVAC systems are controllable by using the data in aggregate.

In yet another embodiment, the sensor system described is used for making smart labels for various shipping and safety applications.

In another embodiment, nanoparticles (e.g., silver) are printed on a substrate (cellulosic or otherwise), and the chemiresistors are printed directly on top of a created circuit. The device is integrated with an RFID, NFC or other similar communication component. By using an external RFID/NFC/etc. reader, relevant compounds and analytes emitted by food being shipped at a given point in time, are interrogated in a minimal or even a zero power method. In other aspects, different electrical and device configurations can be implemented. For example, a low power BLE device could be used to transmit the data actively rather than relying on a passive RFID like device.

In still yet another embodiment, the sensor system is used for personal health applications. A wearable device is used by a subject with respiratory issues ranging from asthma to lung cancer to COPD. By measuring the exposure of the different device users to the individual particles that make up poor air quality, it is possible to reduce the number of incidents as well as the severity of any experienced incidents that they may experience. Further, by providing the device to young children, it is possible to avoid poor air quality analytes, and help them avoid developing respiratory syndromes such as those listed above. In another aspect, a distributed network tracks a plume of poor air given off by a factory, or other point source, and alerts users with the device before it reaches them, and allow them to take precautionary measures.

In another embodiment, a wearable device is used in more stringent medical applications. For example, in diabetics, acetone concentrations are typically much higher than in the breath of non-diabetics. A wearable device of the present invention is used to pre-screen patients for further and more in depth testing. This application is extended to the detection of trace components in a person's breath that may also be of medical interest/concern, which offers insights into diseases ranging from cancer to lactose intolerance.

F. Alternate Form Factors

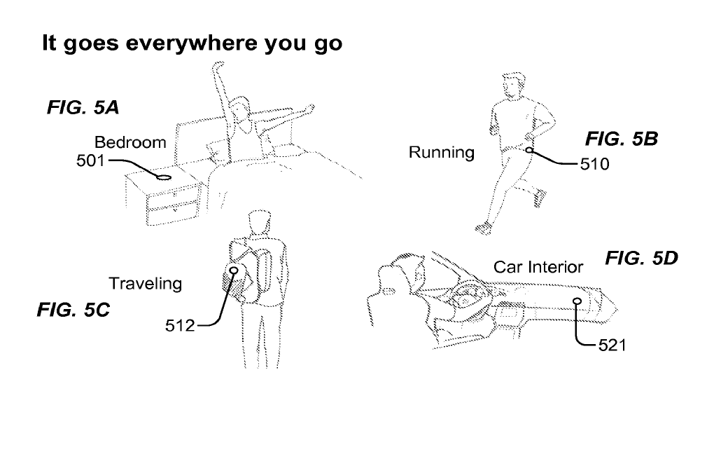

Advantageously, the components of a wearable wristband device can be modified into various shapes to function and fit alternative needs and uses. For example, FIG. 5A shows sensor 501 being used for interrogating the home environment. FIG. 5B shows sensor array 510 being used to interrogate the jogger's environment. FIG. 5C shows sensor array 512 being used to interrogate via a backpack. In addition, FIG. 5D shows the sensor array 521 being used in a mobile application.

In certain aspects, the wearable sensor system is a member selected from the group consisting of a bracelet, a necklace, or a badge. In certain aspects, a single form factor is wearable on different areas of the body. In one example, the core components of a wrist-watch shaped and sized device is taken off the wrist and attached to a worker's belt instead. In an alternative embodiment, a simple strap is added onto the core component and attached to a backpack or other mobile carrying case. In still other instances, the sensor can be worn on a belt, backpack mounted, attached to clothing, suitcases or brief cases.

In still other aspects, the core components of the device are used in conjunction with other pre-existing devices to make a system with new or additional functionalities. In one aspect, the device integrates a particle sensor that detects particles including those classified as PM2.5 or PM10 to provide a more complete picture of air quality. Given the size constraints of the particle sensor, this system's form factor is larger than those described previously, closer in size to a portable box or container like device. These embodiments may be placed on any flat surface, like a desk, or mounted onto a wall or ceiling similar to a smoke detector. Further, this device is used for a wide range of industrial applications, especially in data centers and in automotive applications.

In yet other aspects, the device integrates a particle sensor that detects particles including those classified as PM2.5 or PM10 to provide a complete picture of air quality.

In certain aspects, the wearable sensor system is a member selected from the group consisting of a bracelet, a necklace, a badge and a ring. In certain aspects, the wearable device is a bracelet.

In yet other aspects, the device includes a particle sensor that can detect particle size of 5 micron or below, the system can detect particulate air pollutant and/or allergen detection.

Other applications include those in industrial markets, ranging from the automotive to food quality monitoring. Further applications include monitoring air quality in vehicles, and the HVAC systems within vehices. Suitable applications include monitoring VOCs that are given off by a variety of foods, and freshness monitoring in real time to reduce spoilage rates especially when shipping these foods long distances.

### G. Manufacturing

Manufacture of wearable sensing devices can be divided into phases. One phase, for example, is the synthesis of the of the polymer/carbon black composite for each sensor in the array. In addition, another phase is the design parameters and the fabrication process/assembly of the device as a whole.

i. Material preparation

The first step in synthesizing the polymer/carbon black composite is to dissolve the polymer using commercially available chemical solvents. This generates the composite of the solution that is applied to a substrate. For example, polyvinyl stearate is dissolved using dichloromethane, polyvinyl alcohol is dissolved using boiling-temperature water, poly (4-vinylphenol) is dissolved using pure ethanol, polybutadiene is dissolved using toluene, and PEVA is also dissolved using toluene. In all of the above cases, the solute to solvent ratio is about 0.1 to about 5 mg/ml such as about 0.65 mg/ml. For polyvinyl stearate, the solute to solvent ratio is about 2.31 mg/ml. The higher ratio is due to the relatively high speed by which polyvinyl stearate dissolves in DCM, and the higher amount of polymer in the composite slows degradation and baseline drift over time.

ii. Design Parameters

The resistance of a chemiresistor is an important parameter for the power consumption.

By having each chemiresistor in the 100 kΩ range, the power consumption of the core components of the device remain in the mW range for the whole sensor array. The resistance without being exposed to a chemical and at a fixed temperature of the chemiresistor depends on the polymer-carbon particle composite ratio, electrical and physical properties of each material, dimension of the chemiresistor film and electrode geometry. One effect of the polymer-carbon black composite ratio on the composite resistivity is highly non-linear; there is a critical volume ratio (i.e., percolation threshold) where the resistivity changes dramatically (10 orders of magnitude change in resistivity when carbon particle volume % changes by 1%). A carbon black volume fraction slightly above this threshold gives both good sensor sensitivity (smaller measurement error) and a resistivity range feasible for sensor electronics (resistance of 1-100 kΩ range). Percolation threshold depends on the physical properties of polymer and carbon particle, but typically it is between 0.05-0.3. An estimated resistance of a polymer-carbon composite with 0.2 of percolation threshold using General Effective Medium (GEM) model gives about 150 kΩ ohm when 25 vol % carbon black is used, and the film thickness is 2 μm.

iii. Fabrication Process

The electrodes are deposited onto a substrate by a microfabrication process and then the polymer/carbon black composite-solution is sprayed onto a substrate pre-heated to 100° C. By using a heated substrate, the solvent used to dissolve the composite evaporates fast and the composite bonds to the substrate faster than without pre-heating. Masks to expose or protect structures are used to make the sensor array. Electric connections are connected to a PCB board with microcontrollers, reference resistors and other components such as low-energy Bluetooth.

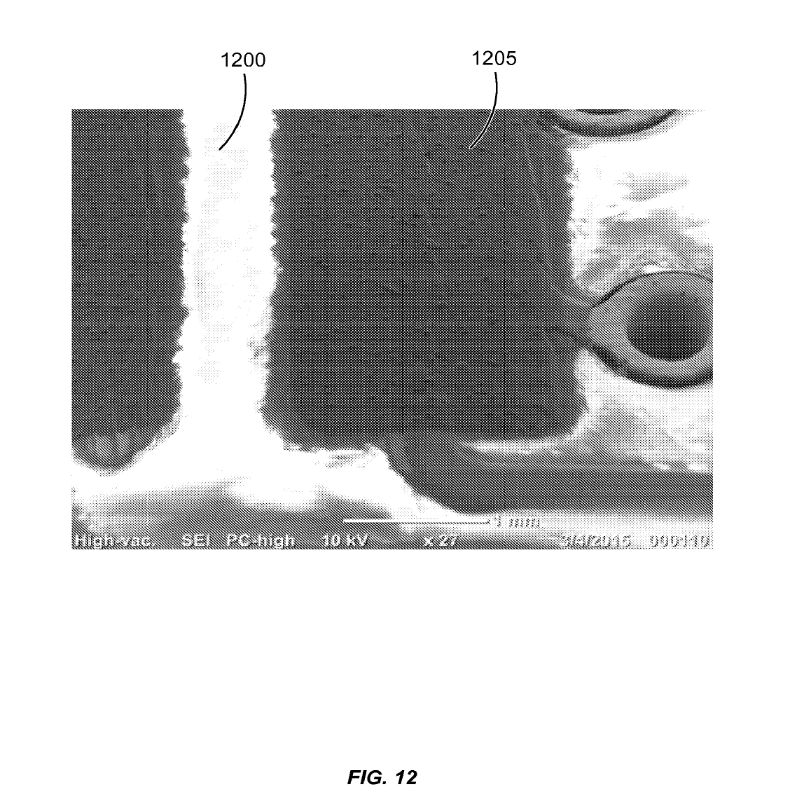

The polymer/carbon inks can be deposited and manufactured in different manners. In one aspect, inkjet printing methodologies are used. By using a thermal inkjet printhead, polymer/carbon composites are deposited onto a given substrate as illustrated in FIG. 12.

Thickness and composition of the deposited film can be modified by altering the viscosity of the inputted inks. Further, by using a piezoelectric printhead, circuits of silver, copper or other metal particles can also be printed or deposited onto the same substrate. In one aspect, a unique sensor tag itself is completely manufactured via printing.

Roll-to-roll manufacturing can also be utilized to produce thin films in larger bulk.

### II. EXAMPLES

### Example 1

Testing is done to determine which chemicals a polymer-carbon composite material is able to detect and also determine how sensitive the chemiresistors are to the analyte in question.

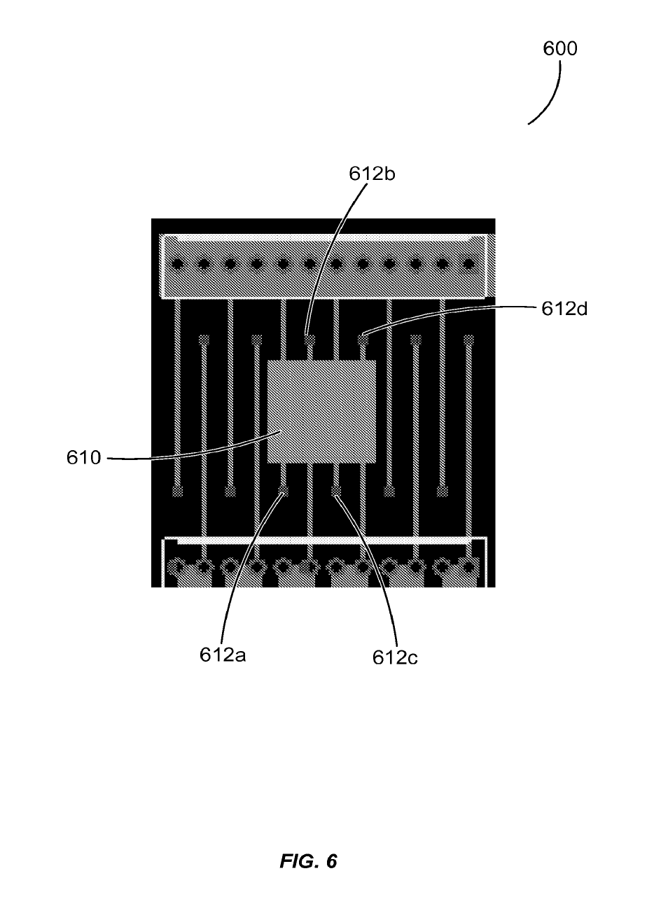

In one example, a centimeter-scale sensor using four polymers is made. Each polymer-carbon composite is sprayed using an airbrush onto a custom-designed PCB board on which there is an array of electrodes. In one embodiment, a 1 cm×1 cm chemiresistor film is on four electrodes, which separation between the two is 2.54 mm and the electrode width is 0.38 mm. FIG. 6 illustrates a chemiresistor 600 having a 1 cm×1 cm square 610 dimensions sprayed onto four electrodes (612a-d). By assigning +V/0/+V/O to each electrode, this one patch can work as three identical chemiresistors.

As shown in FIG. 12, the chemiresistor film 1205 sprayed by an airbrush has a non-uniform thickness profile, but the average thickness is about 200 μm. A separation area 1200 exists between each sensor of the array. The resistance measured across the two center electrodes ranges 1-50 kΩ for each chemiresistor. Then each sensor is connected with a reference resistance across which voltage is connected to an Arduino board input channel to take data. Data acquisition is done by an Arduino-Matlab interface.

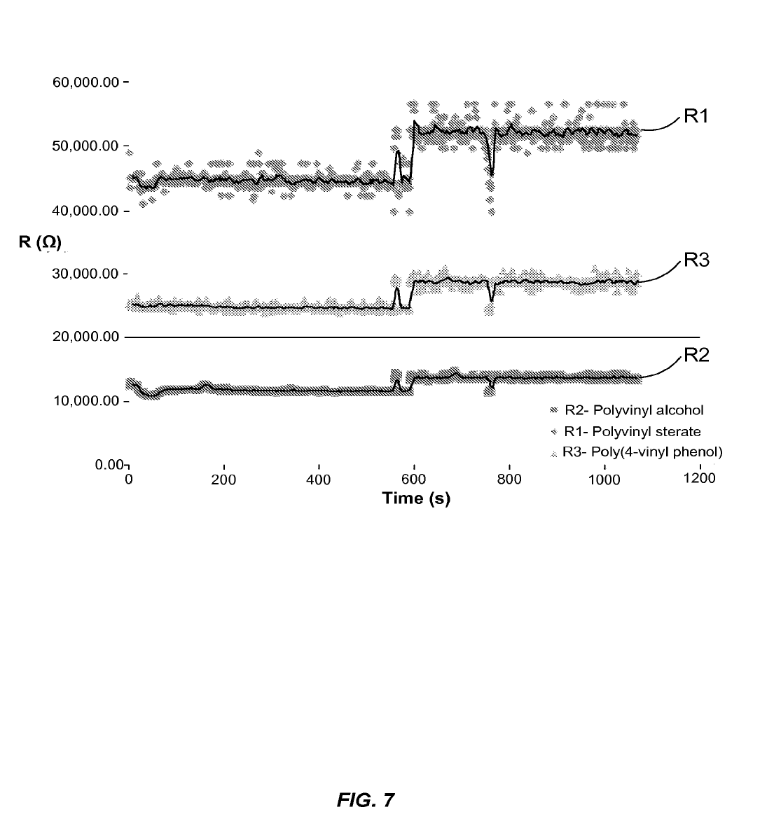

Each test chemical was sprayed on to the sensor array with an airbrush from a half-meter distance. With a presence of chemicals such as ethanol and methanol, the resistance of the sensors changed about 10-15%. As shown in FIG. 7, in the presence of ethanol in the parts per million range, the resistance increased by 15% for the polyvinyl stearate (R1) chemiresistor, 10% for the polyvinyl alcohol (R2) chemiresistor and 15% for the poly (4-vinyl phenol) (R3) chemiresistor. Methanol, a compound in the same alcohol family as ethanol, saw a 10% change in resistance in the polyvinyl stearate and poly (4-vinyl phenol) chemiresistors. In certain aspects, polyvinyl stearate and poly (4-vinyl phenol) are used to detect short to medium carbon chain alcohols.

Other analytes that can be detected include chemicals such as acetic acid and tetrahydrofuran (THF), and human breath. Acetic acid is detectable using a polyvinyl alcohol chemiresistor, which saw a 5% increase in resistance upon exposure. THF is detectable using polyvinyl stearate, which saw a 4% increase in resistance upon exposure. Human breath is detectable by polyvinyl stearate, poly (4-vinyl phenol) and polybutadiene. Polyvinyl stearate and poly (4-vinyl phenol) both show approximately a 10% change in resistance upon exposure and polybutadiene showed a 5% change.

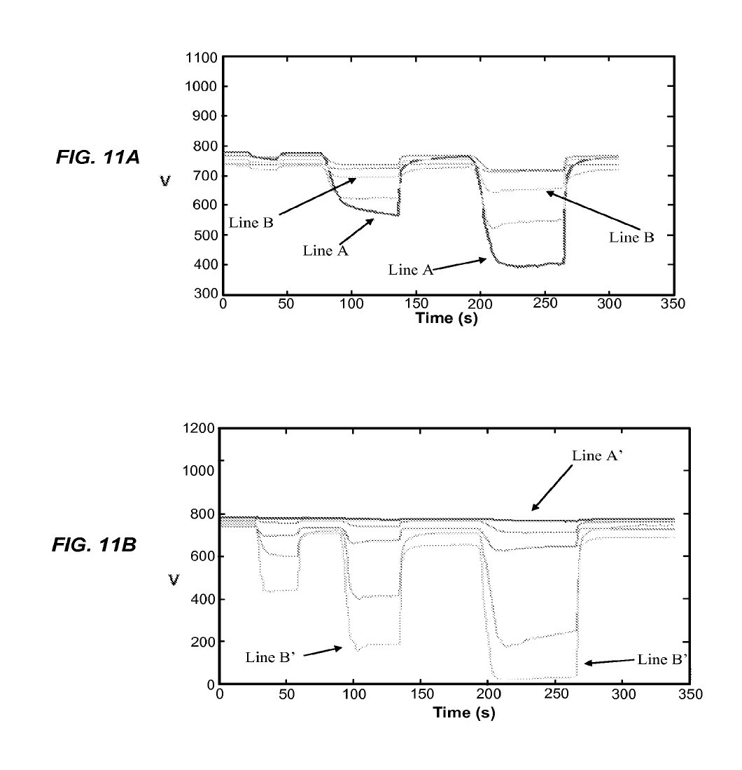

As demonstrated above, different kinds of chemiresistors respond differently to a chemical, and the resulting profile from the combination of the response signal is unique to the chemical. Thus the sensors can be used to identify a specific chemical. For example, as shown in FIG. 11A, a 2×3 array of polymers (the sixth slot was a null control) was exposed to three different but equivalent concentrations of different compounds, including toluene and ethanol. As shown therein, the reaction profiles for these compounds are markedly different. For example, the polymer represented by the line-A (PEVA) saw the largest voltage drop across it when exposed to ethanol (a 400 bit volt drop at highest concentrations) (FIG. 11A). As shown in FIG. 11B, it effectively did not react when exposed to toluene line-A′. By contrast, the polymer represented by line-B′ saw the largest voltage drop when exposed to the highest concentration of toluene (FIG. 11B), but reacted relatively gently as shown by line-B when exposed to even the highest concentrations of ethanol (FIG. 11A). Similar reactions and reaction profiles can be seen across all of the five polymers on the array, and the resulting characteristic reaction can then be used to identify in this case whether toluene or ethanol caused the changes in voltages across the chemiresistors. This identification process can also be facilitated by using pattern recognition software based on principle component analysis (PCA) and/or cluster analysis.

### Example 2

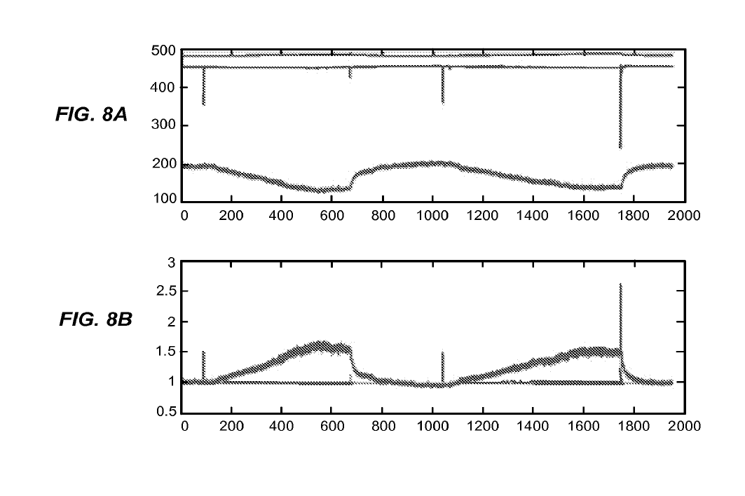

FIGS. 8A-B are representative reaction curves of a sensor array to acetic acid. When the analyte is added into an environmental chamber containing a sensor system, the voltage drop across each of the active chemiresistors (FIG. 8A) rapidly changes before coming to an equilibrium value. When the environmental chamber is purged of the analyte being tested, the resistance returns to its original value (FIG. 8A). This is used to make an equivalent change of resistance plot (FIG. 8B). For the test shown in this figure, the same concentration of acetic acid was added and purged twice directly in succession, resulting in the two humps seen in the FIG. 8B. When exposed to the same concentration, the reaction curves are effectively identical for the same chip. The calculations for converting changes in voltage to the change in resistance is done automatically by software. Converting the raw voltage drops into resistance changes helps significantly ease the pattern recognition process, especially given that the input power and voltages can change across applications. The y axis units is in bit-volts. The vertical spikes indicate when analytes were added and purged from the test chamber. From this, it is clear that the sensors react rapidly to any input and output events.

### Example 3

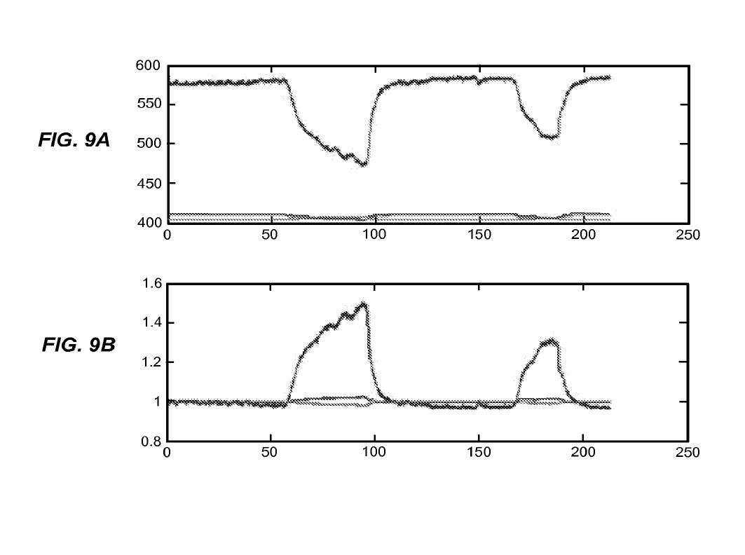

FIGS. 9A-B are representative reaction curves of a sensor array to toluene. The methodology used to run the test that generated the data shown here is the same as described above in Example 2 with a slight modification. For the data shown, two different concentrations of toluene were added and purged twice directly in succession, resulting in the two humps seen in FIG. 9B. The second added concentration of toluene was half that of the first exposure. When the concentration of the analyte the sensor is exposed to is reduced to half, the change in both resistance and voltage of the active components is also roughly halved (compare the height of the humps on the left hand and right hand sides of FIGS. 9A and 9B). This example illustrates the linear response of the sensors to concentration for analytes.

### Example 4

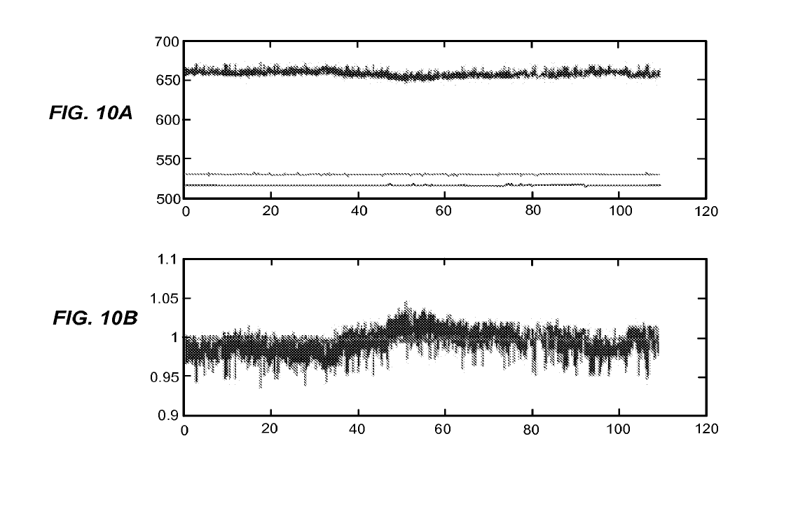

FIGS. 10A-B are representative reaction curves of a sensor array to tetrahydrofuran (THF). Even through the concentration of THF was over 11 times greater than the toluene concentration in FIG. 9, the reaction curve can hardly be discerned from the intrinsic noise of the sensor. Clearly, selectivity can be imparted into the individual sensing elements.

By amalgamating several chemiresistors of different compositions and selectivities onto a single device, the array generates very different characteristic reaction curves for different analytes. For example, despite using the same 5 chemiresistors in FIG. 11A and FIG. 11B, the curves are markedly different for the same concentrations of ethanol and toluene.

### Example 5

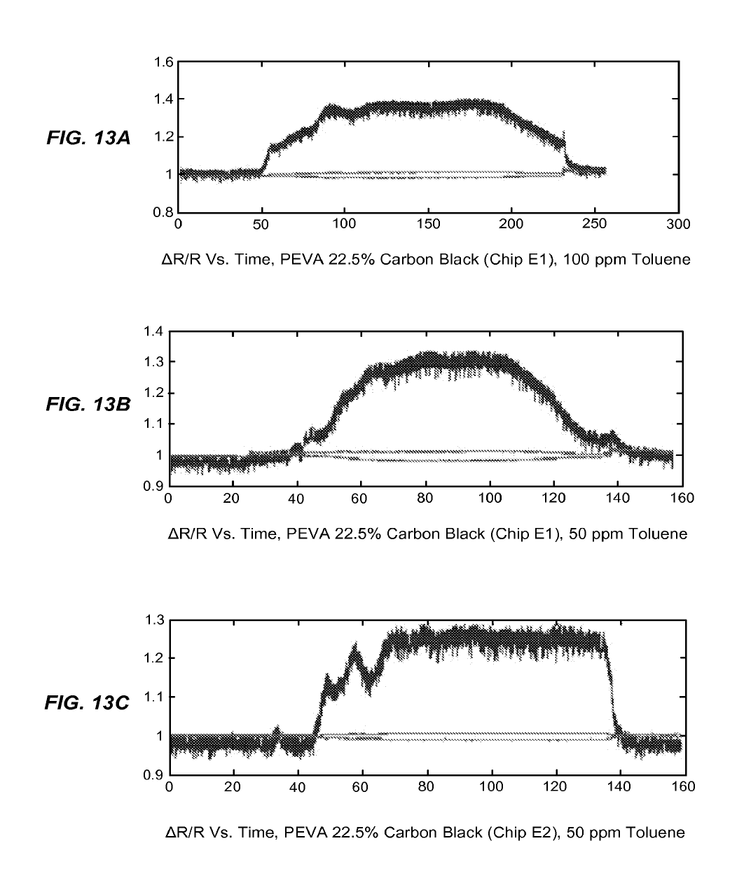

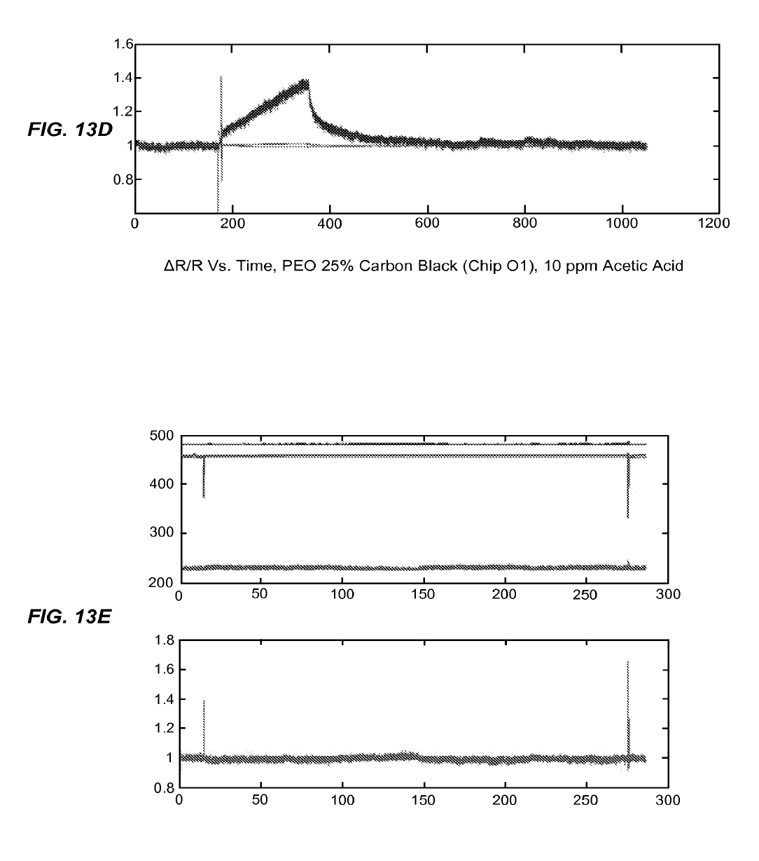

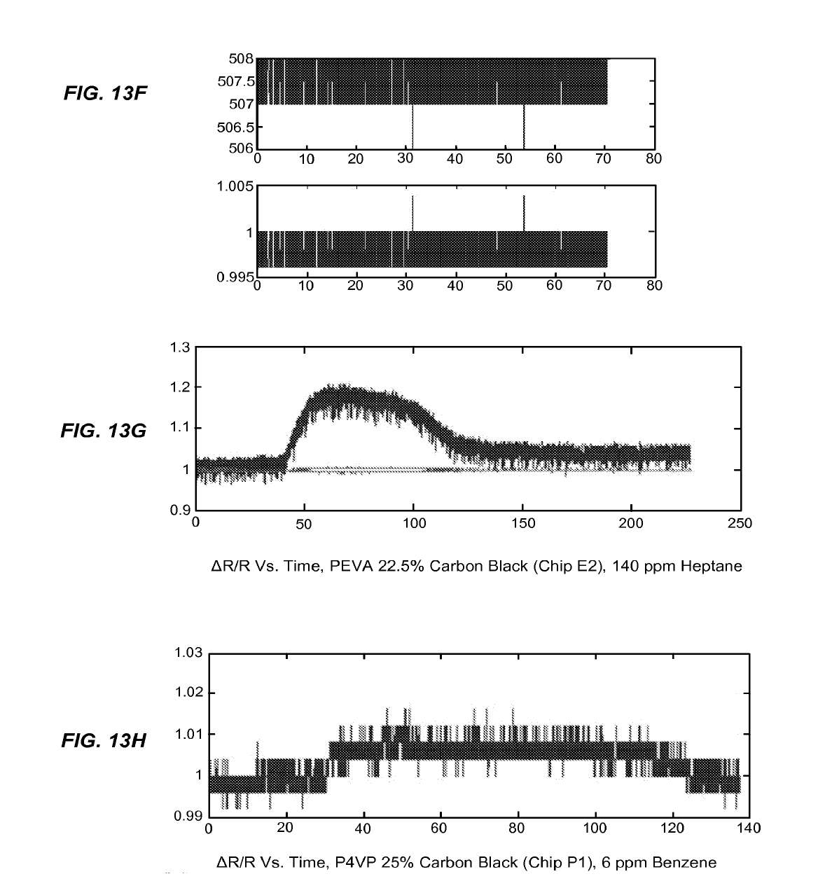

The following data represents how arrays are formed and how the sensors can detect and differentiate different chemicals. FIGS. 13A-H represent a change in resistance with respect to baseline resistance with time. All tests are performed at 30% relative humidity and 20° C., in a test apparatus by dropping and exposing the sensor to a pre-measured volume of analyte and chamber. For volatile organic analytes, the analytes are allowed to evaporate and diffuse in the chamber and then the chamber is evacuated and exposed to pure air.

FIG. 13A illustrates a plot showing a reaction to a carbon-polymer composite to toluene at a concentration of 100 ppm. The carbon-polymer composite consists of Polyethylene-co-vinyl acetate (PEVA) as the polymer and carbon black (23% by weight of polymer). The plot shows a 40% change in resistance with respect to baseline resistance for 100 ppm of toluene. The analyte was introduced inside the testing apparatus at 50 seconds. As illustrated, there is a rise in resistance with evaporation of the analyte and a return to baseline on purging the chamber with pure air at 230 seconds.

Similarly, FIG. 13B illustrates a plot showing a reaction of the same carbon-polymer composite to a lower concentration of toluene at 50 ppm. As seen in FIG. 13B, the reaction to toluene at a concentration of 50 ppm is less than the reaction to toluene at a concentration of 100 ppm. The rise in resistance is only 30% from the baseline, and it returns back to baseline resistance on evacuating the testing apparatus.

All sensors are extremely consistent, as shown in FIG. 13C, which illustrates the reaction curve for a different chip with the same carbon black and polymer blend ratio on exposure to 50 ppm of toluene at a different point of time. FIG. 13C shows a 30% change in resistance on exposure to 50 ppm toluene.

In addition to the detection of the presence of a particular analyte, it is extremely important to differentiate between the presence of different analytes. FIG. 13D shows the reaction of a chip (O1), which is a polymer composite comprising Polyethylene oxide (PEO) and 25% carbon black (by weight) to 10 ppm of glacial acetic acid.

A 35% change in baseline resistance can be seen with exposure of the polymer composite to 10 ppm acetic acid. In contrast, FIG. 13E shows the reaction of the chip (O1) comprising PEO as the polymer on exposure to 200 ppm of toluene. As clearly seen in FIG. 13E, there is no reaction between the polymer PEO and Toluene even in concentrations as high as 200 ppm. The two spikes represent the exposure to toluene and its evacuation from the chamber.

Similarly, FIG. 13F shows the reaction of a chip (E1) containing PEVA as the polymer on exposure to acetic acid at 10 ppm. Once again, there is no reaction between the polymer PEVA and the analyte acetic acid.

FIG. 13G shows the reaction of a chip (E2) containing PEVA as the polymer on exposure to heptane at 140 ppm.

FIG. 13H shows the reaction of a chip (P1) containing Poly-4-VinylPhenol (P4VP) as the polymer on exposure to benzene at 6 ppm.

As illustrated in FIGS. 13A-H, PEO does not react to heptane or toluene while PEVA and Polyvinyl stearate (PVS) react to heptane but do not react to P4VP. Hence, by putting different polymers in an array, it is possible to differentiate between various analytes like heptane, acetic acid, benzene and toluene.

### Example 6

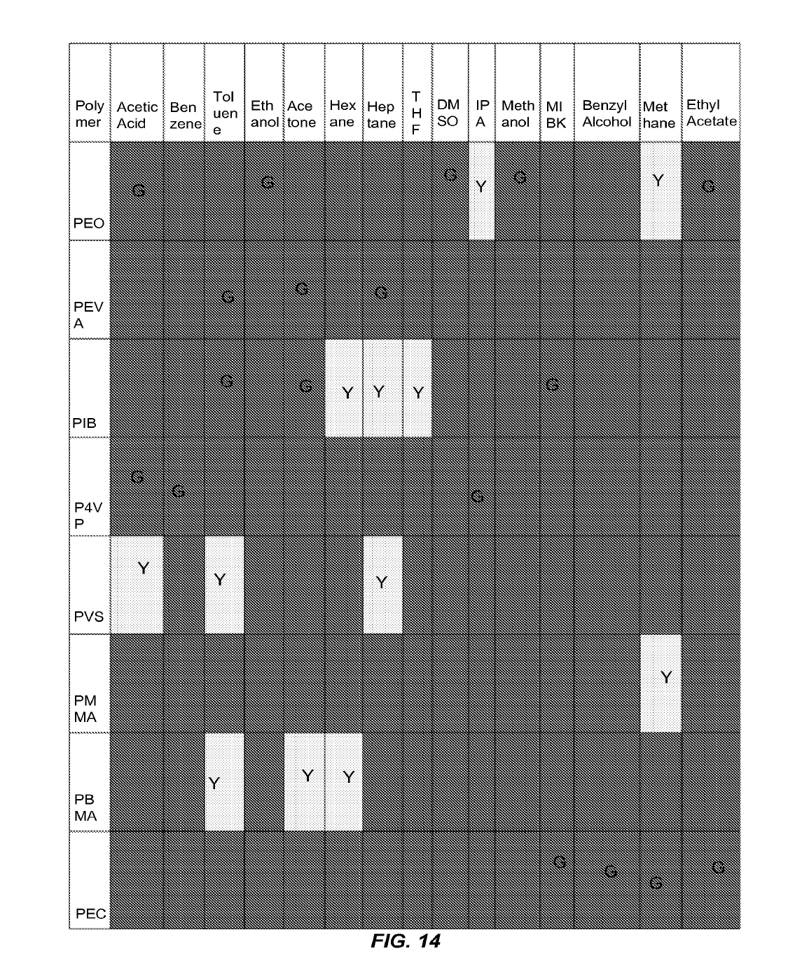

FIG. 14 shows an array in accordance with an embodiment of the present invention, where each row contains a polymer and each column contains an analyte. The “G” boxes represent a polymer's ability to detect a particular analyte with a high response rate and the dark grey boxes show its inability to respond. The “Y” boxes respond with less of a response than the “G” boxes. Each analyte has a different unique fingerprint for an array of polymers.

When identifying potential polymers to detect a desired analyte, prescreening is possible by using a variety of methods, including solvation constants. By using a polymer with a solvation constant very close to the solvation constant of the analyte in question, the likelihood of successful detection increases markedly.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications, websites, and databases cited herein are hereby incorporated by reference in their entireties for all purposes.

What is claimed is:

1. A wearable sensor system for air quality monitoring, the wearable sensor system comprising:
a module comprising an array of chemiresistors;
a microcontroller with a wireless transmitter; and
a signal generator.

2. The wearable sensor system of claim 1, wherein each chemiresistor in the array of chemiresistors comprises a polymer.

3. The wearable sensor system of claim 2, wherein the polymer can be the same or different in each member of the array.

4. The wearable sensor system of claim 2, wherein the polymer is a cellulosic polymer.

5. The wearable sensor system of claim 2, wherein each chemiresistor comprises the polymer and carbon black.

6. The wearable sensor system of any one of claims 1-5, wherein a voltage or an electrical signal pattern from the array of chemiresistors is collected by the microcontroller.

7. The wearable sensor system of claim 6, wherein the voltage or electrical signal pattern is processed by an algorithm to identify a gas.

8. The wearable sensor system of claim 7, wherein the algorithm is resident on the microcontroller.

9. The wearable sensor system of any one of claims 1-8, wherein the sensor system further comprises one or more of a member selected from the group consisting of an accelerometer, a UV-lamp, a micro-heater, a nano-heater, a GPS module, a temperature sensor, a humidity sensor, an RFID tag, and a battery.

10. The wearable sensor system of claim 9, wherein the system comprises an accelerometer.

11. The wearable sensor system of claim 6, wherein the voltage or electrical signal is transmitted from the wearable sensor to a mobile device.

12. The wearable sensor system of claim 7, wherein the identity of the gas is transmitted from the wearable sensor to a mobile device.

13. The wearable sensor system of claim 11 or 12, wherein the transmission from the wearable device to a mobile device is via Bluetooth, cellular, WiFi or other wireless technology.

14. The wearable sensor system of claim 13, wherein the mobile device is a smartphone, cellular phone, tablet or PC.

15. The wearable sensor system of claim 11, wherein the mobile device communicates to a server.

16. The wearable sensor system of claim 15, wherein a plurality of mobile devices communicate to the server.

17. The wearable sensor system of claim 16, wherein an algorithm resides on the server to process the identity of a gas.

18. The wearable sensor system of claim 16, wherein the server generates a localized map of air quality.

19. The wearable sensor system of claim 18, wherein the algorithm is used to estimate a range and concentration of a gas.

20. The wearable sensor system of claim 17, wherein the mobile device receives the identity of the gas from the server.

21. The wearable sensor system of claim 17, wherein the mobile device receives and visualizes a localized map from the server.

22. The wearable sensor system of claim 21, wherein the localized map is overlayed on a user's location.

23. The wearable sensor system of claim 22, wherein a user's movements are transmitted to the server.

24. The wearable sensor system of claim 20, wherein the mobile device transmits the identity of the gas to the wearable sensor system.

25. The wearable sensor system of claim 24, wherein the gas indicates poor air quality.

26. The wearable sensor system of any one of claims 1-25, wherein the signal generator produces a light, a sound, heat, a vibration and a combination thereof

27. The wearable sensor system of any one of claims 1-26, wherein the wearable sensor system is a member selected from the group consisting of a bracelet, a necklace, a badge and a ring.

28. The wearable sensor system of claim 27, wherein the wearable device is a bracelet.

29. The wearable sensor system of claim 27, wherein the bracelet comprises a display screen.

30. The wearable sensor system of claim 27, wherein the bracelet is made of a material selected from the group consisting of a thermoplastic elastomer, a thermoplastic urethane and a silicone rubber.

31. A method of detecting an analyte with a wearable sensor system, the method comprising:
contacting an analyte with a wearable sensor system, the wearable sensor system comprising a module having an array of chemiresistors, a microcontroller with a wireless transmitter, and a signal generator; and
detecting an electrical change in the array of chemiresistors in the presence of the analyte.

32. The method of claim 31, wherein each chemiresistor in the array of chemiresistors comprises a polymer.

33. The method of claim 32, wherein the polymer can be the same or different in each member of the array.

34. The method of claim 32, wherein the polymer is a cellulosic polymer.

35. The method claim 32, wherein each chemiresistor comprises the polymer and carbon black.

36. The method of any one of claims 31-35, wherein a voltage or an electrical signal pattern from the array of chemiresistors is collected by the microcontroller.

37. The method of claim 36, wherein the voltage or the electrical signal pattern is processed by an algorithm to identify a gas.

38. The method of claim 37, wherein the algorithm is resident on the microcontroller.

39. The method of any one of claims 31-38, wherein the sensor system further comprises one or more of a member selected from the group consisting of an accelerometer, a UV-lamp, a micro-heater, a nano-heater, a GPS module, a temperature sensor, a humidity sensor, an RFID tag, and a battery.

40. The method of claim 39, wherein the system comprises an accelerometer.

41. The method of claim 36, wherein the voltage signal is transmitted from the wearable sensor to a mobile device.

42. The method of claim 37, wherein the identity of the gas is transmitted from the wearable sensor to a mobile device.

43. The method of claim 41 or 42, wherein the transmission from the wearable device to a mobile device is via Bluetooth, cellular, WiFi or other wireless technology.

44. The method of claim 43, wherein the mobile device is a smartphone, cellular phone, tablet or PC.

45. The method of claim 41, wherein the mobile device communicates to a server.

46. The method of claim 45, wherein a plurality of mobile devices communicate to the server.

47. The method of claim 46, wherein an algorithm resides on the server to process the identity of a gas.

48. The method of claim 46, wherein the server generates a localized map of air quality.

49. The method of claim 47, wherein the algorithm is used to estimate a range and concentration of a gas.

50. The method of claim 47, wherein the mobile device receives the identity of the gas from the server.

51. The method of claim 47, wherein the mobile device receives and visualizes a localized map from the server.

52. The method of claim 48, wherein the localized map is overlayed on a user's location.

53. The method of claim 52, wherein a user's movements are transmitted to the server.

54. The method of claim 50, wherein the mobile device transmits the identity of the gas to the wearable sensor system.

55. The method of claim 54, wherein the gas indicates poor air quality.

56. The method of any one of claims 31-55, wherein the signal generator produces a light, a sound, heat, a vibration and a combination thereof.

57. The method of any one of claims 31-56, wherein the wearable sensor system is a member selected from the group consisting of a bracelet, a necklace, a badge and a ring.

58. The method of claim 57, wherein the wearable device is a bracelet.

59. The method of claim 57, wherein the bracelet comprises a display screen.

60. The method of claim 57, wherein the bracelet is made of a material selected from the group consisting of a thermoplastic elastomer, a thermoplastic urethane and a silicone rubber.
